(19) United States
(12) Patent Application Publication
(10) Pub. No.: US 2007/0123571 A1
(43) Pub. Date: May 31, 2007

(54) Use of a compound in the treatment of sleep disorders

(76) Inventors: Palaniswamy Sunder Raj, Ettingen (CH); Adrain Shephard, Nottingham (GB); Huw Jones, Nottingham (GB)

Correspondence Address: NIXON & VANDERHYE, PC, 901 NORTH GLEBE ROAD, 11TH FLOOR, ARLINGTON, VA, 22203, US

(21) Appl. No.: 10/557,196

(22) PCT Filed: Jun. 1, 2004
(86) PCT No.: PCT/GB04/02330

§ 371 Date: Apr. 12, 2006

(30) Foreign Application Priority Data
May 30, 2003 (GB) 0312419.5

(51) Int. Cl. A61K 31/4439

(52) U.S. Cl. 514/343

(57) ABSTRACT

There is disclosed the use of triprolidine, in combination with at least one further active pharmaceutical agent, for enabling an individual to wake refreshed after sleep and the method of treating such an individual with triprolidine. Use of triprolidine, in combination with at least one further active pharmaceutical agent, as active ingredient in the manufacture of a composition for the treatment of sleep disorders is also described. A method of treating sleep of a person suffering from a sleep disorder, which method comprises administration of an effective dose of triprolidine, in combination with at least one further active pharmaceutical agent, as active i ingredient to such a person is also described. The triprolidine is administered shortly before a person wishes to fall asleep, preferably orally and most commonly in the form of a tablet containing up to 20 mg, e.g. 0.1 mg, 1.25 mg or 2.5 mg, of the active ingredient. The triprolidine is also effective in enabling an individual to sleep more easily.

The invention relates to a novel use of a known compound, in particular to the use of that compound in combination with at least one further active pharmaceutical agent in the treatment of sleep disorders experienced by a person, whatever the cause of those disorders.

The present invention also relates to a method for the treatment or prevention of grogginess, drowsiness or lethargy on waking from sleep, to the use of triprolidine in combination with at least one further active pharmaceutical agent as an aid to waking refreshed and to the use of triprolidine in combination with at least one further active pharmaceutical agent as both a sleep aid and a means to wake refreshed thereafter.

Although much is known about the use of various pharmaceutical sleeping formulations as aids to sleeping, little has been published about the possibility of a sleep aid enabling an individual to wake refreshed as opposed to merely experiencing degrees of hangover effects such as grogginess, drowsiness, lethargy, etc.

Many people experience, either on an occasional or chronic basis, difficulty in achieving a satisfactory amount of sleep. Such a problem may be attributable to external factors, such as factors causing stress or anxiety, to excessive use or misuse of stimulants (such as caffeine) or depressants (e.g. alcohol), or to temporary disturbance of the person's lifestyle, e.g. occasioned by shift-working or long-haul travel through different timezones. Difficulty in sleeping may also be caused by chronic pain, e.g. pain caused by sciatica, etc. Whatever the cause, the condition may be generally considered to be a sleep disorder and may commonly be referred to as “insomnia”. It may manifest as difficulty in falling asleep and/or wakefulness during the desired period of sleep, leading to a shortened duration of sleep and/or disruption of the normal pattern of sleep.

The result of these difficulties will commonly be fatigue during the period of wakefulness, which may itself lead to stress and exacerbate the problem.

Various products are available to assist a user in overcoming problems of the type described above. Such products, commonly called “sleeping pills” may, however, suffer from disadvantageous side-effects. For example, while the products may be effective in sending a user to sleep, their effect may be of short duration, resulting in premature wakening. In other cases, the user may achieve the desired length of sleep but may awake with feelings of grogginess (a “hangover” effect). Such products may also be addictive. Tolerance may also develop to the drug which results in a decrease in effectiveness.

In other circumstances, a person may not suffer from sleep disorders as such, but may simply wish to achieve a particularly good night's sleep. In other words, the use of such products may be elective, rather than necessitated by a clinical need.

In addition to this well documented problem, many people also experience difficulties on waking such as grogginess, lethargy and drowsiness; difficulty in becoming fully alert and an absence of feeling refreshed. These phenomena are not necessarily linked to the number of hours sleep or always encountered as a result of drugs taken prior to sleep such as alcohol, medication, etc.

Furthermore, individuals encountering tiredness during waking hours and other individuals having difficulty with insomnia resort to sleep aids in an attempt to increase or improve sleeptime rest. Nevertheless, it is also well documented that a negative side effect of sleep aids can also be an increased feeling of grogginess on waking.

Triprolidine, (E)-2-[1-(4-methylphenyl-3-(1-pyrrolidinyl)-1-propenyl]pyridine, is a first generation anti-histamine and has been marketed alone and, in combination with pseudoephedrine (a decongestant), for the treatment of allergic rhinitis. Triprolidine is known to have sedative effects and has been shown to have an adverse effect on the cognitive functions of users. These are undesirable side-effects for an anti-histamine and may account for the limited extent to which triprolidine has been used in clinical practice. More recently-developed, second generation anti-histamines are less prone to such side effects, and most recent studies involving triprolidine have used that compound as a positive control against which the more modern anti-histamine compounds have been compared. Such studies have generally been conducted using healthy volunteers following day time dosing, rather than persons suffering from any form of sleep disorder, and have been concerned with the effects of the drug on day-time performance.

One study is known to have investigated the effect of triprolidine (amongst other anti-histamines) on sleep directly (Nicolson et al, Neuropharmacology (1985) 24, 3, 245-250). In that study single doses of triprolidine (10 mg or 20 mg sustained release) were given at bedtime to volunteers. It was found that triprolidine did not significantly alter “sleep onset latency” (i.e. the time required to fall asleep) compared with placebo. It was also found that, compared with placebo, triprolidine had no effect on wakefulness during sleep or total sleep time.

It has now been found that, contrary to what might have been expected in the light of previous studies, triprolidine can be used for inducing, prolonging or enhancing sleep, and that its use is accompanied by important benefits in comparison with other compounds known for this purpose that could not have been predicted.

It has also been found that triprolidine surprisingly increases the level of refreshedness felt upon waking if taken before sleeping. Advantageously, this effect is observed whilst triprolidine also acts as a sleep aid in facilitating the onset of stage I sleep and whilst enhancing sleep.

The increased level of refreshedness felt upon waking after taking triprolidine prior to sleeping was not expected and there has been no known disclosure of such an effect previously encountered.

In many medical conditions, lack of sleep is experienced as a side effect or direct symptom of the medical condition. Often, a patient with such a condition will be prescribed sleep aids as well as being treated for the specific medical condition.

According to a first aspect of the present invention there is provided the use of triprolidine or a salt or hydrate thereof, in combination with at least one further active pharmaceutical agent, as active ingredient of an aid to waking refreshed after sleeping.

According to a second aspect of the present invention there is provided the use of triprolidine or a salt or hydrate thereof, in combination with at least one further active pharmaceutical agent, as active ingredient for the preparation of a composition for enabling an individual to wake refreshed after sleeping.

According to a third aspect of the present invention there is provided the use of triprolidine or a salt or hydrate thereof, in combination with at least one further active pharmaceutical agent, as active ingredient for the preparation of a medicament for enabling an individual to wake refreshed after sleeping.

According to a fourth aspect of the present invention there is provided the use of triprolidine or a salt or hydrate thereof, in combination with at least one further active pharmaceutical agent, for the preparation of a sleep aid which also enables an individual to wake refreshed after sleeping.

According to a fifth aspect of the present invention there is provided the use of triprolidine or a salt or hydrate thereof, in combination with at least one further active pharmaceutical agent, as active ingredient of a sleep aid which also enables an individual to wake refreshed after sleeping.

According to a sixth aspect of the present invention there is provided the use of triprolidine or a salt or hydrate thereof, in combination with at least one further active pharmaceutical agent, as active ingredient for the preparation of a medicament for the treatment or prevention of a sleep disorder which also enables an individual to wake refreshed after sleeping.

According to a seventh aspect of the present invention there is provided a method for the treatment or prevention of grogginess, drowsiness or lethargy on waking from sleep in a mammal comprising the administration to the mammal in need thereof of a non-toxic effective dose of triprolidine or a salt or hydrate thereof in combination with at least one further active pharmaceutical agent prior to the desired sleeping time.

According to an eighth aspect of the present invention there is provided a method for enabling an individual to wake refreshed after sleeping comprising the administration to the individual in need thereof and prior to the desired sleeping time of a non-toxic effective dose of triprolidine or a salt or hydrate thereof in combination with at least one further active pharmaceutical agent.

According to a ninth aspect of the present invention there is provided a method for aiding an individual's sleep and for also enabling the individual to subsequently wake refreshed after sleeping comprising the administration to the individual in need thereof and prior to the desired sleeping time of a non-toxic effective dose of triprolidine or a salt or hydrate thereof in combination with at least one further active pharmaceutical agent.

According to a tenth aspect of the present invention there is provided a waking refreshed aid comprising triprolidine or a salt or hydrate thereof, in combination with at least one further active pharmaceutical agent, as active ingredient in association with a pharmaceutically acceptable carrier therefor and instructions for administration thereof at or just before the desired sleeping time.

According to an eleventh aspect of the present invention there is provided a pharmaceutical formulation for the treatment or prevention of grogginess, drowsiness or lethargy on waking after sleeping, comprising triprolidine or a salt or hydrate thereof, in combination with at least one further active pharmaceutical agent, as active ingredient in association with a pharmaceutically acceptable carrier therefor and instructions for administration thereof at or just before the desired sleeping time.

According to a twelfth aspect of the present invention there is provided a pharmaceutical formulation for enabling an individual to wake more refreshed after sleeping, comprising triprolidine or a salt or hydrate thereof, in combination with at least one further active pharmaceutical agent, as active ingredient in association with a pharmaceutically acceptable carrier therefor and instructions for administration thereof at or just before the desired sleeping time.

According to a thirteenth aspect of the present invention there is provided a method of treating sleep of a person suffering from a sleep disorder, which method comprises administration of an effective dose of triprolidine, in combination with at least one further active pharmaceutical agent, as active ingredient to such a person.

According to a fourteenth aspect of the present invention, there is provided the use of triprolidine, in combination with at least one further active pharmaceutical agent, as active ingredient in the manufacture of a composition for the treatment of sleep disorders.

According to a fifteenth aspect of the invention, there is provided a method for inducing, prolonging and/or enhancing sleep, which method comprises administration of an effective dose of triprolidine, in combination with at least one further active pharmaceutical agent, as active ingredient to a person desirous of achieving sleep.

In a related aspect of the invention, there is provided the use of triprolidine as active ingredient thereof in combination with at least one further active pharmaceutical agent in the manufacture of a composition for inducing, prolonging and/or enhancing sleep.

The invention extends to a kit comprising a first pharmaceutically active dosage form having triprolidine as the active agent, a second pharmaceutically active dosage form and instructions on how to administer the said first and second dosage forms.

The said first and second dosage forms may be located in separate compartments of a pharmaceutical pack.

The said dosage forms may be combined into a combined dosage form for simultaneous administration.

Preferably, the said at least one further active pharmaceutical agent is intended to be used in the treatment of a condition having sleep disorder as a symptom or potential symptom.

Preferably, the said further active pharmaceutical agent may include, without limitation, antacids, analgesics, anti-inflammatories, antibiotics, laxatives, anorexics, antivirals, antiasthmatics, antidiuretics, antiflatulents, antimigraine agents, antispasmodics, additional sedatives, antihyperactives, tranquilizers, antihistamines, decongestants, betablockers, antidepressives, hormones and combinations thereof. More preferably, the further active pharmaceutical agent is an active agent for treatment of pain, allergic conditions, migraine, coughing, a cold, flu, viral infections, throat infection, stress.

Preferably, the said further active pharmaceutical agent is independently intended for use as a, or in the treatment of pain, allergic reactions, migraines, coughs, anaesthetics, antiviral agents, disinfectant, anxiety, decongestant or women's health (such as menopausal or period problems).

Preferably, the said at least one further active pharmaceutical agent is independently selected from: an active agent used in the treatment of pain relief, migraines, allergies, colds, flu, coughs, anxiety, or women's health; an active agent used as an anaesthetic, antiviral agent, decongestant or disinfectant.

More preferably, the active agent is selected from an active agent used in the treatment of pain relief, allergies, anxiety, migraines, colds, flu, coughs and as a decongestant or antiviral agent.

Most preferably, the active agent is selected from an agent used in the treatment of colds, coughs, pain relief and flu.

Preferably, the said at least one further active agent is independently selected from a group consisting of Ibuprofen, Fluribiprofen, Ketoprofen, aspirin, Paracetamol, Aceclofenac, Codeine, Naproxen, Indomethacin, Diclofenac, Cox II, Meloxicam, Nitric oxide, Caffeine, Acrivastine, Cetirizine, Loratadine, Fexofenadine, Terfenadine, Beclomethasone, Hydrocortisone, Triptans, Almotriptan, Rizatriptan, Naratriptan, Sumatriptan, Zolmatriptan, Domperidone, Acetylcysteine, Menthol, Ambroxol, Carbocisteine, Dextromethorphan, Guaiphenesin, Ipecacuanha, Phenylpropanolamine, Liquorice, Marshmallow, Squill, Honey, Glycerine, Aniseed, Benzocaine, Lidocaine, Amantadine, Aciclovir, Famciclovir, Ganciclovir, Rimantadine, Penciclovir, Tribavirin, Valaciclovir, Neuraminidase inhibitors, Zanamir, Oseltamir, Benzalkonium chloride, Cetylpyridinium chloride, Dichlorobenzyl alcohol (dcba), Amylmetacresol(amc), Dequalinium chloride, Hexylresorcinol, Eucalyptus oil, Thymol, Calamine, Propranalol, Chamomile, Hops, Passion flower, Valarian, Melatonin, Eucalyptus, Phenylepherine, Pseudoephedrine, Cranberry and Bisphosphonates or a pharmaceutically acceptable salt of any of the foregoing.

A more preferred range of active agents is independently selected from a group consisting of Ibuprofen, Fluribiprofen, Cox II such as meloxicam, triptans, Domperidone, Ambroxol, Dextromethorphan, Guaiphenesin, Lidocaine, Amantadine, Hexylresorcinol, dcba, amc, Propranalol, pseudoephedrine and Bisphosphonates or a pharmaceutically acceptable salt of any of the foregoing.

Optionally, the further active pharmaceutical agent may be combined with triprolidine in a single dosage form or in a pharmaceutical pack containing at least two dosage forms, one being triprolidine and the other being the said further active pharmaceutical agent. Preferably, the said pack includes instructions on how to take and/or mix the combination of triprolidine with the said further active pharmaceutical agent.

Preferably, the dosage of the said further pharmaceutically active agent is one suitable for the treatment selected. Preferably, a single dosage form of said pharmaceutically active agent is in the range 0.1 mg-2000 mg, more preferably, 0.2 mg-1000 mg, most preferably, 0.5 mg-100 mg.

Typically, the dosage form for a pharmaceutical active in the treatment of pain is in the range 1-2000 mg, more preferably, 5-1000 mg depending upon the suitable dose level of the further active pharmaceutical agent.

Typically, the dosage form for a pharmaceutical active in the form of triptans is in the range 0.1-200 mg, more preferably, 0.5-100 mg depending upon the suitable dose level of the further active pharmaceutical agent.

Typically, the dosage form for a pharmaceutical active in the treatment of viral infections is in the range 1-1000 mg, more preferably, 50-300 mg depending upon the suitable dose level of the further active pharmaceutical agent.

Typically, the dosage form for a pharmaceutical active in the treatment of allergies is in the range 0.1-500 mg, more preferably, 0.5-200 mg depending upon the suitable dose level of the further active pharmaceutical agent.

Typically, the dosage form for a pharmaceutical active in the treatment of coughs and colds is in the range 0.1-500 mg, more preferably, 1-200 mg depending upon the suitable dose level of the further active pharmaceutical agent.

Typically, the dosage form for a pharmaceutical active in the treatment of upper respiratory tract problems is in the range 0.1-100 mg, more preferably, 0.5-50 mg depending upon the suitable dose level of the further active pharmaceutical agent.

Typically, the dosage form for a pharmaceutical active in the treatment of anxiety is in the range 0.1-200 mg, more preferably, 1-100 mg depending upon the suitable dose level of the further active pharmaceutical agent.

It will also be understood that the term “inducing, prolonging and/or enhancing sleep” may encompass the treatment of a sleep disorder, i.e. a difficulty in achieving satisfactory sleep due to some internal or external factor, e.g. pain, stress or anxiety, misuse of stimulants or depressants, or temporary disturbance of lifestyle. Alternatively, it may encompass elective desires on the part of a user to achieve a particularly beneficial period of sleep. Such a desire may, for instance, arise in anticipation of important events the following day for which a person may wish to be fully alert and refreshed. In any event, the term “sleep disorder” as used herein should be taken to independently include any one or more of the foregoing and, specifically, any objective or subjective difficulty in an individual in any one or more of the following:—
getting to sleep, especially stage 1 sleep
staying asleep
sleeping well
waking refreshed
waking alert
keeping awake
keeping alert
keeping refreshed
performing well the next day

The present invention also extends to the use of triprolidine as a sleep aid. By definition, a sleep aid extends to use by a healthy individual who elects for a sleep aid, for example, before an important event. The term “sleep aid” as used herein includes any one or more of the following benefits:—
faster onset to stage 1 sleep
increasing duration of sleep periods
decreasing the number and duration of awakenings
increasing total duration of sleep
increasing probability of sleeping well
improving insomnia, especially chronic or mild-moderate insomnia
decreasing disturbances during sleeptime
improving quality of sleep,
as determined by any standard or known subjective or objective measures, for instance the Karolinska scale, Loughborough sleep log, Leeds sleep evaluation questionnaire or actimetry.

The method of aiding an individual's sleep typically indicates aiding in the sense of providing any one or more of the above mentioned benefits.

Typically, the percentage of individuals who, after taking a dose of triprolidine before sleeptime, wake refreshed after sleeping is in the range 1-100%, more typically, 5-70%, most typically 10-35%. An especially typical range as aforesaid is 15-30% or even more especially 20-30%. Typically, by the terms “waking refreshed” or “wake refreshed” is meant that an individual felt at least refreshed on waking, preferably, the terms are defined as the individual felt very refreshed or refreshed in accordance with the Loughborough sleep log.

Typically, the percentage of individuals who, after taking a dose of triprolidine before sleeptime, wake refreshed after sleeping is more than 2%, more typically, more than 8% and most typically, more than 15%. An especially typical level as aforesaid is more than 18% or even more especially more than 20%.

By the term sleeping as referred to herein is meant an individual in at least Stage I sleep. By the term sleeptime as referred to herein is meant the time an individual desires to go to sleep.

Typically, the percentage of individuals who, after taking a dose of triprolidine before sleeptime, felt alert after sleeping is in the range 1-100%, more typically, 5-60%, most typically 10-30%. An especially typical range as aforesaid is 15-30% or even more especially 20-30%.

Typically, the percentage of individuals who, after taking a dose of triprolidine before sleeptime, felt alert after sleeping is more than 2%, more typically, more than 8%, most typically more than 12%. An especially typical level as aforesaid is more than 16%.

By the term felt alert is meant that an individual felt at least alert on waking. Preferably, the term is defined as the individual felt alert, very alert or extremely alert in accordance with the Karolinska 9-point scale.

Typically, the percentage of individuals who, after taking a dose of triprolidine before sleeptime, felt sleepy on waking is less than 25%, more typically, less than 20%, most typically less than 15%. An especially typical level as aforesaid is less than 14% or even more especially a mean level of less than 12%.

By the term felt sleepy is meant that an individual felt sleepy on waking. Preferably, the term is defined as the individual felt sleepy or very sleepy in accordance with points 8 or 9 of the Karolinska 9-point scale.

Preferably, in use of the present invention as defined herein, the mean subjective feeling of refreshedness after waking as, for instance, determined on a 5 point scale, e.g. by the morning log of the Loughborough sleep log, is increased by at least 2%, more typically, by at least 4%, most typically, by at least 5%, as compared with an equivalent dose of placebo.

Typically, in use of the present invention as defined herein, the mean subjective feeling of refreshedness after waking as for instance, determined on a 5 point scale, e.g. by the morning log of the Loughborough sleep log, is increased by between 1-20%, more typically, 1-15%, most typically 2-10% as compared with an equivalent dose of placebo.

The degree of refreshedness and quality of sleep may be determined by the “morning” log of the Loughborough sleep log with the highest degree of refreshedness or quality of sleep being represented as 1 and the lowest being represented as 5. Accordingly, the percentage increase in refreshedness or quality of sleep is measured in this context by the decrease in the mean refreshedness or quality of sleep.

Preferably, by the use of the present invention, the response of awakening very refreshed or refreshed, as determined, for instance, by the morning log of the Loughborough sleep log, is improved by at least 20%, more preferably, by at least, 30%, most preferably by at least 40%, as compared with an equivalent dose of placebo.

Typically, by the use of the present invention, the response of awakening very refreshed or refreshed, as determined, for instance, in accordance with the morning log of the Loughborough sleep log is improved by between 5% and 100%, more typically, by between 10% and 80%, most typically by between 20% and 60%, especially 40-55% and more especially 40-45% as compared with an equivalent dose of placebo.

Preferably, by the use of the present invention, the response of feeling extremely alert, very alert or alert as determined, for instance, in accordance with the Karolinska 9-point scale, is improved by at least 2%, more preferably, by at least, 5%, most preferably by at least 10%, as compared with an equivalent dose of placebo.

Typically, by the use of the present invention, the response of feeling extremely alert, very alert or alert, as determined, for instance, in accordance with the Karolinska 9 point scale, is improved by between 1% and 40%, more typically, by between 2% and 30%, most typically by between 10% and 20%, as compared with an equivalent dose of placebo. An especially preferred range is 10-30%.

Preferably, by the use of the present invention, the response of feeling sleepy and needing to make some effort to stay awake or very sleepy, as determined, for instance, in accordance with points 8 and 9 of the Karolinska 9 point scale, is improved (i.e. decreased) by at least 2%, more preferably, by at least, 4%, most preferably, by at least 10%, as compared with an equivalent dose of placebo.

Typically, by the use of the present invention, the response of feeling sleepy and needing to make some effort to stay awake or very sleepy, as determined, for instance, in accordance with points 8 and 9 of the Karolinska 9 point scale is improved (i.e. decreased) by between 1% and 100%, more typically, by between 2% and 75%, most typically, by between 4% and 60%, as compared with an equivalent dose of placebo.

Preferably, in use of the present invention as defined herein, the sleeptime awakenings, as for example determined by the Night diary of the Loughborough sleep log, may be decreased by 2-40%, typically, by 10-35%, most typically by 15-30%, as compared with an equivalent dose of placebo. An especially preferred range is 15-40%. Preferably, in use of the present invention as defined herein, the sleeptime awakenings may be decreased by more than 5%, more preferably by more than 10%, most preferably, by more than 15%, as compared with an equivalent dose of placebo.

Preferably, in use of the present invention as defined herein, sleep disturbance index (SDI), as for instance determined by actimetry, may be decreased by more than 5%, more preferably by more than 10%, most preferably by more than 15% as compared with an equivalent dose of placebo.

Preferably, in use of the present invention as defined herein, SDI may be decreased by 5-30%, more typically 5-25%, most typically 10-20% as compared with an equivalent dose of placebo. An especially preferred range is 10-30%, more especially 10-25%.

Preferably, in use of the present invention as defined herein, time to sleep onset (TTSO) as, for instance, determined by actimetry may be decreased by 5-40%, more typically 15-35%, most typically 20-30% as compared with an equivalent dose of placebo. An especially preferred range is 20-40%, more especially 20-35%.

Preferably, in use of the present invention as defined herein, the time to sleep onset (TTSO) as compared with an equivalent dose of placebo is decreased by at least 10%, more preferably by at least 15%, most preferably, by at least 20%.

Preferably, the quality of sleep experienced as felt after awakening is also improved by the use of the present invention, typically the quality of sleep is improved by 2-30%, more typically 5-30%, most typically 10-20% as compared with an equivalent dose of placebo and as, for instance, determined by the morning log of the Loughborough sleep log. Typically, in use of the present invention as defined herein, the quality of sleep is improved by at least 2%, more preferably at least 5%, most preferably at least 10% as compared with an equivalent dose of placebo.

Preferably, in use of the present invention, the time to fall asleep as determined, for instance, by the Night diary of the Loughborough sleep log is decreased by 1-40%, more typically 5-35%, most typically 10-30%. An especially preferred range is 10-40%, more especially 10-35%. Typically, in use of the present invention as defined here, the time to fall asleep as aforementioned is decreased by at least 2%, more typically, by at least 5%, most typically by at least 10% as compared with an equivalent dose of placebo.

Preferably, by the use of the present invention, the response of sleeping extremely well or very well, as determined, for instance, in accordance with the morning log of the Loughborough sleep log, is improved by at least 20%, more preferably, at least, 35%, most preferably at least 50%, as compared with an equivalent dose of placebo.

Preferably, by the use of the present invention, the response of sleeping extremely well or very well, as determined, for instance, in accordance with the morning log of the Loughborough sleep log, is found for at least 20% of individuals, more preferably, at least 25%, most preferably, at least 30%. For example over 35% of individuals had such a response.

Typically, by the use of the present invention, the response of sleeping extremely well or very well, as determined, for instance, in accordance with the morning log of the Loughborough sleep log is improved by between 10% and 200%, most typically, by between 20% and 150%, more typically by between 25% and 135% as compared with an equivalent dose of placebo. Typically, by the use of the present invention, the response of sleeping extremely well or very well, as determined, for instance, in accordance with the morning log of the Loughborough sleep log is found for between 25% and 100% of individuals, more typically, 30-80% most typically 35-70%. Especially preferred is the response in at least between 35-60%, of individuals, more especially 35-45%.

It will be understood that references herein to “triprolidine” include the compound (E)-2-[1-(4-methylphenyl-3-(1-pyrrolidinyl)-1-propenyl]pyridine as well as salts thereof that are acceptable for administration to the human body. Acid addition salts may particularly be mentioned, including the hydrobromide and hydrochloride salts. The hydrochloride salt, i.e. triprolidine hydrochloride, is particularly preferred for use in accordance with the invention. Solvates of triprolidine, notably hydrates, e.g. monohydrates, and to the extent that triprolidine may exist in polymorphic forms, all such polymorphs are within the scope of the invention.

The term “refreshed” as used herein means an individual waking refreshed or alert after a dose of triprolidine has been administered prior to sleep. In this context, the determination of whether an individual is feeling “refreshed” may be made by a subjective test. An example subjective test is measuring the degree of alertness on, for instance, the Karolinska scale or the feeling of being refreshed as determined by, for instance, the Loughborough sleep log. Alternatively, refreshedness may be based upon the inverse relationship between refreshedness and relative levels of sleepiness as determined by the Karolinska scale.

By the term individual as referred to herein is meant any mammal or human.

The administration of the active ingredient in accordance with the invention may be beneficial in that there is evidence that users feel more refreshed upon awakening, which is not the case with other treatments for sleep disorders, or indeed in the absence of any treatment, and do not experience grogginess or a “hangover” effect after the required number of hours sleep. This too is surprising in view of the fact that such feelings have been reported in relation to other active ingredients which have a comparable mode of action to that of triprolidine. Furthermore, there is no evidence that repeated use of the active ingredient over the course of several days leads to any loss of effect.

The administration of the active ingredient in accordance with the invention may also be beneficial in that it may decrease the time required for a user to fall asleep, which is surprising in view of the previously-reported studies on volunteers. In addition, the total period of sleep may be increased and the incidence and duration of night-time wakenings experienced by the user may be reduced.

The active ingredients are preferably formulated in such a manner as to lead to non-sustained, substantially immediate release of the active ingredient, i.e. the formulation is preferably free of ingredients intended or effective to prolong or sustain release of the active ingredient.

Administration of the active ingredient in accordance with the invention may be by a variety of routes. However, most commonly the active ingredient will be administered orally. An alternative mode of administration may be administration to the mucous membranes of the nasal passages. Further modes of administration are transdermal (e.g. using transdermal patches or bandages), rectal (e.g. as suppositories), optical, sub-lingual, buccal and pulmonary.

For oral administration, the active ingredient may be put up in a variety of dosage forms. Most commonly, the active ingredient will be formulated and administered as a tablet or the like. However, formulation as capsules, lozenges, drinks or as a syrup (solution or suspension) may also be possible, as may other dosage forms such as a consumable film for instance a buccal wafer or oral sprays.

For nasal administration, the active ingredient may be formulated as a solution, emulsion or suspension and administered by means of a spray using a suitable delivery device. Alternatively, for pulmonary administration, the active ingredient may be administered as a powder, either from a pressurised aerosol delivery device or from a so-called dry powder inhaler.

For formulation in the presently preferred form, i.e. as a tablet, the active ingredient will generally be combined with various excipients in a manner which is known per se. In particular, the tablet will generally comprise one or more diluents or bulking agents. A diluent may also serve as a disintegrant, or the formulation may incorporate a separate disintegrant. A lubricant may also be included to facilitate release of the formed tablets from the tabletting dies of a tablet forming machine.

Thus, according to a further aspect of the invention, there is provided a tablet for enabling an individual to wake refreshed after sleeping, which tablet comprises triprolidine or a salt or hydrate thereof, in combination with at least one further active pharmaceutical agent as active ingredient in admixture with one or more diluents and/or a disintegrant, the tablet comprising more than 0.01 mg and less than 4.9 mg triprolidine.

As noted above, the formulation may incorporate one diluent or bulking agent, or more than one. Formulations are preferred which contain blends of two or more diluents, one of which may also serve as a disintegrant.

Preferred materials for the diluent or bulking agents include polysaccharides and derivatives thereof, and saccharides.

Polysaccharides which may be used include starch, e.g. maize starch, cellulose, e.g. powdered cellulose and microcrystalline cellulose, water-insoluble modified starches, e.g. sodium carboxymethyl starch, water-insoluble cellulose derivatives, e.g. croscarmellose sodium (cross-linked sodium carboxymethyl cellulose), cross-linked polyvinylpyrrolidone and alginic acid.

Another preferred form of diluent is a saccharide. Suitable saccharides include, for example, sucrose, lactose, dextrose, sorbitol, mannitol, xylitol and maltodextrin. Lactose and sucrose are preferred saccharides. Lactose is especially preferred. Saccharide diluents may also be beneficial in terms of modifying the taste of the formulation.

Particularly preferred diluents are dicalcium phosphate, microcrystalline cellulose, e.g. the products sold as Avicel PH-101 and Avicel PH-102 (Avicel is a Trade Mark) by the FMC Corporation of Philadelphia, Pa., USA, and lactose.

Another preferred disintegrant is a croscarmellose sodium, for example the product sold as Ac-Di-Sol (Ac-Di-Sol is a Trade Mark) by the FMC Corporation. This product, when included in the formulation, also serves as a disintegrant.

The disintegrant has the effect of causing the tablet composition to disintegrate under the conditions found in the gastro-intestinal tract. Apart from croscarmellose sodium, examples of disintegrants include one or more of wheat starch, maize starch, potato starch, sodium starch glycolate, low-substituted hydroxypropyl cellulose, alginic acid, cross-linked polyvinylpyrrolidone and magnesium aluminium silicate. Preferred disintegrants are those which swell on the action of water thus causing the ingredients in the tablet to be pushed apart and out into the aqueous disintegration medium. The preferred disintegrant is croscarmellose sodium. The disintegrant is present at an effective disintegrating amount, for example up to 25% by weight of the composition, more preferably 1-25% w/w, further preferably 3-20% w/w and most preferably 5-15% by weight of the composition.

Particularly preferred compositions, in a particular tablet compositions, include a blend of a cellulosic diluent, a saccharide diluent and a disintegrant. The preferred cellulosic diluent is microcrystalline cellulose, the preferred saccharide is lactose and the preferred disintegrant is croscarmellose sodium.

A preferred formulation, in particular a tablet formulation, comprises the cellulosic diluent, the saccharide diluent and the disintegrant in the ratio of 0.01-10 parts by weight of cellulosic diluent, 0.01-10 parts by weight of saccharide diluent to 1 part by weight of disintegrant. More preferably, the formulation contains 2-5 parts by weight of cellulosic diluent per part by weight of disintegrant, and 4 to 7 parts by weight of saccharide diluent per part by weight of disintegrant.

The diluents and/or disintegrant are preferably incorporated into the compositions in finely divided (powder) form.

The diluents and disintegrant preferably together constitute in excess of 80% w/w of the tablet formulation, more preferably in excess of 90% w/w, and most preferably in excess of 94% w/w.

The lubricant may be, for example, stearic acid, a metallic stearate, a polyethylene glycol of molecular weight of 4,000 or more, or purified talc. The preferred lubricant is a metallic stearate, particularly magnesium stearate, which may be present in the formulation at relatively low levels, typically less than 1% or 0.5% by weight.

It has been found to be particularly advantageous for the tablet formulation to be formed with a coating, preferably a sugar coating or film coating process, more preferably a film coating comprising a hydrophilic polymer, particularly a cellulose derivative such as a methylated cellulose derivative, e.g. hydroxyethylmethylcellulose and, particularly, hydroxypropylmethylcellulose.

The coating may also comprise an inorganic filler material, most preferably French chalk, to enhance the physical properties of the coating and prevent cracking etc, and also a pigment, e.g. a titanium dioxide pigment dispersion.

It has been found that, in addition to improving the appearance of the tablet and acting as a barrier to ingress of moisture, the film coating is also effective in masking the taste of the active ingredient.

Administration of the active ingredient in accordance with the invention may be by means of a consumable film. The films may be edible and upon disintegration, the triprolidine and other active may be absorbed via the buccal cavity or the digestive tract. Preferably, the triprolidine and other active are formulated to be absorbed via the digestive tract. Suitable formulations are disclosed in WO 00/18365, the content of which insofar as it relates to consumable film formulations which may incorporate triprolidine hydrochloride or methods of producing such formulations is incorporated herein by reference.

For consumable film formulation in the presently preferred form, the active ingredient will generally be combined with various excipients in a manner which is known per se.

Suitable excipients for consumable films are disclosed in WO 00/18365 and these are incorporated herein by reference.

Thus, according to a further aspect of the invention, there is provided a consumable film for enabling an individual to wake refreshed after sleeping, which film comprises triprolidine as active ingredient in combination with at least one further active pharmaceutical agent in admixture with one or more suitable excipients, the film comprising more than 0.01 mg and less than 4.9 mg triprolidine. The film is preferably, substantially free from menthol, thymol, methyl salicylate and eucalyptol.

The consumable film is one adapted to adhere and dissolve in a mouth of a consumer and comprises at least one water soluble polymer. Preferably, the said water soluble polymer is selected from the group consisting of pellulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

Preferably, other film excipients may be utilised and these may be selected from water, antimicrobial agents, additional film-forming agents, plasticizing agents, flavouring agents, sulphur precipitating agents, saliva stimulating agents, buffering agents, cooling agents, surfactants, stabilising agents, emulsifying agents, thickening agents, binding agents, colouring agents, sweeteners, fragrances and the like.

Saliva stimulating agents can also be added as film excipients. Saliva stimulating agents include food acids such as citric, lactic, malic, succinic, ascorbic, adipic, fumaric and tartaric acids. Preferred food acids are citric, malic and ascorbic acids. The amount of saliva stimulating agents in the film is from about 0.01 to about 12 wt %, preferably about 1 wt % to about 10 wt %, even more preferably about 2.5 wt % to about 6 wt %.

Buffering agents include salts of the aforementioned acids such as alkali metal salts of the food acids detailed above. An especially preferred buffering agent is sodium citrate. The amount of buffering agent may be in accordance with that suitable to complement the saliva stimulating agent as detailed above but is typically 0.01-12 wt %.

Preferred plasticizing agents for the films include triacetin in amounts ranging from about 0 to about 20 wt %, preferably about 0 to 2 wt %. Other suitable plasticizing agents include monoacetin and diacetin.

Preferred cooling agents for the films include monomethyl succinate, in amounts ranging from about 0.001 to 2.0 wt %, preferably about 0.2 to about 0.4 wt %. A monomethyl succinate containing cooling agent is available from Mane. Inc. Other suitable cooling agents include WS3, WS23, Ultracool II and the like.

Preferred surfactants for the films include mono and diglycerides of fatty acids and polyoxyethylene sorbitol esters, such as, Atmos 300 and Polysorbate 80. The surfactant can be added in amounts ranging from about 0.5 to about 15 wt %, preferably about 1 to about 5 wt % of the film. Other suitable surfactants include pluronic acid, sodium lacryl sulphate, and the like.

Preferred stabilising agents for the films include xanthan gum, locust bean gum and carrageenan, in amounts ranging from about 0 to about 10 wt %, preferably about 0.1 to about 2 wt % of the film. Other suitable stabilising agents include guar gum and the like.

Preferred emulsifying agents for the films include triethanolamine stearate, quaternary ammonium compounds, acacia, gelatin, lecithin, bentonite, veegum and the like, in amounts ranging from about 0 to about 3 wt %, preferably about 0.01 to about 07 wt % of the film.

Preferred thickening agents for the films include methylcellulose, carboxylmethylcellulose, and the like, in amounts ranging from about 0 to about 20 wt %, preferably about 0.01 to about 5 wt %.

Preferred binding agents for the films include starch, in amounts ranging from about 0 to about 10 wt %, preferably about 0.01 to about 2 wt % of the film.

Suitable sweeteners for the films that can be included are those well known in the art and similarly, flavourings and colourings that can be included are those known in the art. A suitable definition of sweeteners, flavourings and colourings is found in WO 00/18365, page 12 line 17-page 16 line 19, the contents of which are hereby incorporated herein by reference.

The tablet formulation may be prepared by a process involving dry blending or wet or dry granulation. However, it is preferred to use a manufacturing method which involves direct compression into a tablet without an intermediate, e.g. a wet or dry granulation, stage.

The formulation may be made by dry mixing the active ingredient with the other ingredients, e.g. the lubricant and diluents and disintegrant, e.g. in a powder blending machine. It is particularly preferred that the active ingredients are dispersed by progressive dilution with agitation in a proportion, e.g. about one-half, of the excipients so as to achieve even distribution of the active ingredient in the excipients, and then to add the remainder of the excipients with further agitation and mixing. The mixture may then be compressed in a tablet forming machine and a coating, preferably a sugar coat or a film coat may then be applied to the tablets so formed by spraying the tablets with a solution or suspension of the coating-forming ingredients while the tablets are tumbled.

Such a direct tablet compression manufacturing method has been found to be beneficial in that it avoids problems attributable to crystal growth and changes in morphology which might occur in a wet granulation process.

Other, currently less preferred, dosage forms may be prepared in a manner which is generally known per se. For example, syrups may be prepared by dissolving or suspending the active ingredient in a liquid vehicle, e.g. water, optionally with suspending agents or the like, e.g. cellulose derivatives, gums etc.

For administration by inhalation, via nose or mouth, the formulations may be formulated with a compressed gas or liquified gas propellant, e.g. any conventionally used propellant such as a chlorofluorocarbon, hydrofluorocarbon, compressed hydrocarbon, nitrogen etc. Alternatively, the active ingredient may be formulated as a dry powder, generally in admixture with a diluent such as crystalline lactose.

The amount of active ingredient to be administered in a single dose may vary quite widely, depending inter alia on the desired effect and the mode of administration. However, a formulation for oral administration, e.g. a tablet, will generally contain at least 0.01 and up to 20 mg of active ingredient, more commonly at least 0.5 mg and less than 10 mg of active ingredient, most commonly no more than 5 mg, e.g. 1.25 or 2.5 mg. Doses of formulations for administration by nasal and sub-lingual administration, which would be expected to deliver the active ingredient more quickly and efficiently, may contain less active ingredient, e.g. between 0.1 and 11.0 mg, e.g. about 0.5 mg and generally at a level of 20% of the oral dose levels mentioned herein. Preferably, such nasal and sub-lingual formulations contain active ingredient in the range 0.01-2.5 mg, more preferably, 0.05-11.0 mg and most preferably, 0.1-0.5 mg.

In general, the desired dose (which may comprise one or more unit doses, e.g. one or two tablets or the like) will be taken by a user prior to the desired time at which it is desired for the composition to take effect. Most commonly, the dose will be taken at night-time, i.e. prior to the user sleeping through hours of darkness. Typically, the dose may thus be taken after 8 pm in the evening or later, say after 9 pm or after 10 pm. Typically, it may be recommended that the user take the composition between 0, more commonly 1 minute and 2 hours prior to the time at which he or she wishes to fall asleep. Most commonly, the composition may be taken about 10 to 30 minutes prior to that time. In addition, however, the active ingredient may be effective, particularly at lower doses, in restoring sleep, e.g. in the event of night-time waking.

Preferably, the use of triprolidine in any aspect of the invention as defined herein is its use as active ingredient. Preferably, the triprolidine in any aspect of the invention defined herein is in the form of a non-toxic effective dose, preferably, suitable for any given mammal or human and determined in accordance with age and weight.

Preferably, to obtain the benefits on waking or otherwise as defined herein, the active ingredient of triprolidine administered before sleeptime is less than 10 mg, typically less than 5 mg, more preferably, less than 4.5 mg, most preferably less than 4.0 mg. Especially preferred is a dose as aforesaid of less than 3.5 mg and most especially preferred is a dose of less than 3.0 mg. Typically, the dose of triprolidine is between 0.01 and 10.0 mg, preferably, between 0.01 and 4.9 mg, more preferably, between 0.1 and 4.5 mg, most preferably between 0.5 and 4 mg. Especially preferred is a dose of between 1 and 3.5 mg and more especially a dose of between 2.0 and 3.0 mg. Most especially preferred is a dose as aforesaid of about 2.5 mg or 1.25 mg. Preferably, the above dosage levels are based on triprolidine hydrochloride monohydrate and amounts of other salts or hydrates should be varied accordingly to deliver the equivalent amount of active ingredient.

In the formulations of the present invention, the triprolidine may be in any suitable release form such as a slow release, sustained release, immediate release or uncontrolled release form. The formulation may also be in any one or more of the following delivery forms:—

Pastilles

lozenge

chewable tablets

fondant-fill tablets

coated or uncoated tablets

sub-lingual tablets

fast-melt tablets

hot or cold drinks

syrups

drops

emulsions

dry powder

suspension

transdermal patch

suppository

consumable films such as buccal wafers

sub-lingual and nasal sprays

Preferably, the dose of the triprolidine and further active agent in accordance with the invention may be taken by an individual before it is desired to go to sleep (sleeptime), preferably less than two hours before sleeptime, more preferably, less than one hour before sleeptime, most preferably, less than 20 minutes before sleeptime. Especially preferred is to take the dose of triprolidine and further active agent less than 15 minutes before sleeptime.

Preferably, the dose of triprolidine and further active agent is less than 4 doses per day (24 hour period), more preferably, less than 3 doses per day, most preferably less than 2 doses per day. Especially, preferred is 1 dose per day.

The packaging of the invention as defined herein may be in any suitable form such as, for example, a blister pack, bottle, tamper-proof container, sachet, box, etc. The packaging of the invention may be associated with instructions for any of the features or preferred features of the invention as defined herein.

For the avoidance of doubt, reference to the “use of the present invention” herein should be taken to include “the method of the invention”, and “use of a pharmaceutical formulation” as well as use of the present invention per se.

Advantageously, the use of triprolidine and further active agent in the present invention results in a reduced hangover or morning grogginess effect as compared with other sleep aids or sleep disorder remedies. More advantageously, the use of triprolidine and further active agent in the present invention provides an improved degree of refreshedness or more refreshed feeling upon waking as determined by the Loughborough sleep log, Leeds sleep evaluation questionnaire or Karolinska scale and as compared with placebo.

For the avoidance of doubt, reference to quantities of triprolidine herein should be taken as references to quantities of the hydrochloride mono hydrate (HCl. H₂O) form. However, it should be appreciated that the invention extends to other forms, including all pharmaceutically active salts and hydrates thereof.

The term refreshed as used herein may be substituted by any term selected from alert, invigorated, revitalised, re-energised, recharged, rejuvenated, attentive, awake or words having the like effect or equivalent general meaning and the term refreshedness may also be substituted by the grammatical equivalent thereof from the words aforesaid. In addition, the term alert as used herein can be substituted by any of the above alternative terms.

Non limiting embodiments of the invention will now be illustrated with reference to the accompanying examples.

Experimental

The dosage forms were prepared as tablets, lozenges and syrups as follows.

Tablet Manufacture

Sieve the lactose, pregelatised maise starch, maize starch, ac-di-sol and active materials into a granulator mixer and mix for 5 minutes. In a side vessel prepare the granulating solution using plasdone and water. Add this solution to the granulator, until a suitable granule is formed. Dry the granule in a fluid bed dryer and sieve. Sieve the magnesium stearate through a 30 mesh sieve and add to the granule and blend for 2 minutes. Compress the blend to the appropriate tablet weight.

Lozenge Manufacture

Sieve the calcium carbonate and active materials through a 30 mesh sieve into a granulator mixer. Mix for 5 minutes.

In a side vessel prepare the granulating solution using plasdone and water. Add this solution to the granulator, until a suitable granule is formed.

Dry the granule in a fluid bed dryer and sieve. Sieve the aerosil and magnesium stearate through a 30 mesh sieve and add to the granule and blend for 2 minutes.

The base solution (sugar and glucose) is pumped into the pre-cooker and heated to 114 C+/−5 C to increase the solids content from approximately 72% solids to approximately 85% solids. The heated mass is then pumped to the main cooker and further heated to 140o C.+/−5 C to achieve a solids content of approximately 96% solids. A vacuum of 0.8+/−0.1 of a bar is then applied to achieve a mass having a solids content of approximately 98%. The hot mass is discharged continuously into a mixing chamber. Flavour and the active granule are dosed into the cooked mass at a rate to meet the finished product composition, given the flow rate of the cooked mass. The mixed mass is continuously discharged from the mixing chamber, passed down a tempering belt, cooled and collected in the batch former. The mass is drawn into a rope and passed through a drop former. Lozenge weight checks are made at regular intervals. The lozenges pass through a cooling conveyor which operates within the temperature range of 12-25 C before being collected into storage containers.

Syrup Manufacture

In a suitable stainless steel manufacturing vessel the hydroxyethylcellulose is dispersed in 2300 litres of liquid sucrose.

The mixture is then homogenised until smooth and lump free. The remaining 700 litres of liquid sucrose is then added to the bulk along with 500 litres of purified water and mixed until homogenous. The mixture is then left to stand for 2 hours to allow the hydroxyethylcellulose to hydrate.

In a suitable stainless steel manufacturing vessel the glycerol is warmed to 55-60° C. and the active materials added and mixed until dissolved. This is then added to the hydroxyethylcellulose/liquid sucrose bulk mixture with stirring. The glycerin vessel is then rinsed with 100 litres of purified water that is also added to the bulk vessel. The mixture is then stirred until homogenous.

The citric acid, sodium citrate and sodium saccharin are then added directly to the bulk solution and stirred until dissolved. The colouring ingredients are dissolved in 10 litres of purified water in a suitable stainless steel vessel before being added to the bulk solution with mixing. The vessel is rinsed with 10 litres of purified water that is also added to the bulk mixture with stirring.

The levomenthol, domiphen bromide and flavours are mixed in 80 litres of ethanol 96% in a suitable stainless steel vessel. The solution is added, with stirring to the bulk mixture that has been pre-cooled to below 32° C. The flavouring manufacturing vessel is then rinsed with 20 litres of ethanol 96% that is then also added to the bulk mixture with stirring.

Final Bulk Production

The bulk mixture is made up to final volume with purified water and stirred for 30 minutes to ensure homogeneity. An in-process viscosity check is performed at this point.

Examples of tablet formulations which may be used in the invention are as follows:

### EXAMPLES

Pain
Tablet Formulae (mg/tab)

| | | | | Pregelatinised | | | Magnesium | | Tablet wt |
|---|---|---|---|---|---|---|---|---|---|
| | | Triprolidine HCl | Lactose | Maize Starch | Maize Starch | Ac-di-sol | Stearate | Plasdone K-29-32 | (mg/) |
| Ibuprofen | 200 | 2.5 | 95.5 | 12 | 48 | 25 | 2 | 15 | 400 |
| Ibuprofen | 400 | 2.5 | 95.5 | 12 | 48 | 25 | 2 | 15 | 600 |
| Flurbiprofen | 50 | 2.5 | 145.5 | 12 | 48 | 25 | 2 | 15 | 300 |
| Dexketoprofen | 12.5 | 2.5 | 183 | 12 | 48 | 25 | 2 | 15 | 300 |
| Diclofenac Sodium | 75 | 2.5 | 145.5 | 12 | 48 | 25 | 2 | 15 | 325 |
| Celecoxib | 200 | 2.5 | 95.5 | 12 | 48 | 25 | 2 | 15 | 400 |
| Indomethacin | 50 | 2.5 | 145.5 | 12 | 48 | 25 | 2 | 15 | 300 |
| Ketoprofen | 100 | 2.5 | 145.5 | 12 | 48 | 25 | 2 | 15 | 350 |
| Mefenamic acid | 500 | 2.5 | 148 | 12 | 48 | 25 | 2 | 15 | 750 |
| Naproxen | 250 | 2.5 | 95.5 | 12 | 48 | 25 | 2 | 15 | 450 |
| Rofecoxib | 12.5 | 2.5 | 183 | 12 | 48 | 25 | 2 | 15 | 300 |
| Piroxicam | 20 | 2.5 | 175.5 | 12 | 48 | 25 | 2 | 15 | 300 |
| Tenoxicam | 20 | 2.5 | 175.5 | 12 | 48 | 25 | 2 | 15 | 300 |
| Aspirin | 500 | 2.5 | 148 | 12 | 148 | 25 | 2 | 15 | 750 |
| Paracetamol | 500 | 2.5 | 148 | 12 | 48 | 25 | 2 | 15 | 750 |

Lozenge Formulae (mg/loz)

| | | | | | | Liquid | Liquid | | | | |
|---|---|---|---|---|---|---|---|---|---|---|---|
| | | Triprolidine | | Magnesium | Calcium | Glucose (sol | Sugar (sol | | Water | Plasdone | Lozenge |
| | | HCl | Aerosil | stearate | Carbonate | contents) | contents) | Flavour | (ml) | K29-32 | wt (mg/) |
| Ibuprofen | 200 | 2.5 | 0.05 | 0.249 | 150 | 700 | 1241 | 7.05 | 47 | 1..5 | 2350 |
| Ibuprofen | 400 | 2.5 | 0.05 | 0.249 | 150 | 600 | 1141 | 7.05 | 47 | 1.5 | 2350 |
| Flurbiprofen | 8.75 | 2.5 | 0.05 | 0.249 | 7.5 | 1010 | 1266 | 7.05 | 47 | 1.5 | 2350 |
| Dexketoprofen | 12.5 | 2.5 | 0.05 | 0.249 | 10 | 1010 | 1196 | 7.05 | 47 | 1.5 | 2350 |
| Diclofenac Sodium | 75 | 2.5 | 0.05 | 0.249 | 70 | 800 | 1390 | 7.05 | 47 | 1.5 | 2350 |
| Celecoxib | 200 | 2.5 | 0.05 | 0.249 | 150 | 700 | 1241 | 7.05 | 47 | 1.5 | 2350 |
| Indomethacin | 50 | 2.5 | 0.05 | 0.249 | 50 | 850 | 1342 | 7.05 | 47 | 1.5 | 2350 |
| Ketoprofen | 100 | 2.5 | 0.05 | 0.249 | 75 | 825 | 1292 | 7.05 | 47 | 1.5 | 2350 |
| Mefenamic acid | 500 | 2.5 | 0.05 | 0.249 | 150 | 500 | 1142 | 7.05 | 47 | 1.5 | 2350 |
| Naproxen | 250 | 2.5 | 0.05 | 0.249 | 7.5 | 680 | 1354 | 7.05 | 47 | 1.5 | 2350 |
| Rofecoxib | 12.5 | 2.5 | 0.05 | 0.249 | 7.5 | 1010 | 1196 | 7.05 | 47 | 1.5 | 2350 |
| Piroxicam | 20 | 2.5 | 0.05 | 0.249 | 15 | 950 | 1307 | 7.05 | 47 | 1.5 | 2350 |
| Tenoxicam | 20 | 2.5 | 0.05 | 0.249 | 15 | 950 | 1307 | 7.05 | 47 | 1.5 | 2350 |
| Aspirin | 500 | 2.5 | 0.05 | 0.249 | 150 | 500 | 1142 | 7.05 | 47 | 1.5 | 2350 |
| Paracetamol | 500 | 2.5 | 0.05 | 0.249 | 150 | 500 | 1142 | 7.05 | 47 | 1.5 | 2350 |

Syrup Formulae (mg/5 ml)

| | | | Glyc- | Liquid | Hy- | | | Sodium | Fla- | Ethanol | Levo- | Domiphen | | |
|---|---|---|---|---|---|---|---|---|---|---|---|---|---|---|
| | | Triprolidine | erol | Sucrose | droxyethyl | Citric | Sodium | Sac- | vour | 96% | men- | Hydro- | Col- | |
| | | HCl | (ml) | (ml) | cellulose | Acid | Citrate | charin | (ml) | (ml) | thol | bromide | our | Water |
| Ibuprofen | 200 | 2.5 | 0.9 | 2.9 | 12.5 | 17 | 50 | 12.5 | 0.009 | 0.1 | 1 | 0.25 | 0.9 | to 5 ml |
| Ibuprofen | 400 | 2.5 | 0.8 | 2.8 | 12.5 | 17 | 50 | 12.5 | 0.009 | 0.1 | 1 | 0.25 | 0.9 | to 5 ml |
| Flurbiprofen | 8.75 | 2.5 | 0.99 | 2.99 | 12.5 | 17 | 50 | 12.5 | 0.009 | 0.1 | 1 | 0.25 | 0.9 | to 5 ml |
| Dexketoprofen | 12.5 | 2.5 | 0.99 | 2.99 | 12.5 | 17 | 50 | 12.5 | 0.009 | 0.1 | 1 | 0.25 | 0.9 | to 5 ml |
| Diclofenac | 75 | 2.5 | 0.95 | 2.95 | 12.5 | 17 | 50 | 12.5 | 0.009 | 0.1 | 1 | 0.25 | 0.9 | to 5 ml |
| Sodium | | | | | | | | | | | | | | |
| Celecoxib | 200 | 2.5 | 0.9 | 2.8 | 12.5 | 17 | 50 | 12.5 | 0.009 | 0.1 | 1 | 0.25 | 0.9 | to 5 ml |
| Indomethacin | 50 | 2.5 | 0.95 | 2.95 | 12.5 | 17 | 50 | 12.5 | 0.009 | 0.1 | 1 | 0.25 | 0.9 | to 5 ml |
| Ketoprofen | 100 | 2.5 | 0.95 | 2.95 | 12.5 | 17 | 50 | 12.5 | 0.009 | 0.1 | 1 | 0.25 | 0.9 | to 5 ml |
| Mefenamic | 500 | 2.5 | 0.75 | 2.75 | 12.5 | 17 | 50 | 12.5 | 0.009 | 0.1 | 1 | 0.25 | 0.9 | to 5 ml |
| acid | | | | | | | | | | | | | | |
| Naproxen | 250 | 2.5 | 0.9 | 2.9 | 12.5 | 17 | 50 | 12.5 | 0.009 | 0.1 | 1 | 0.25 | 0.9 | to 5 ml |
| Rofecoxib | 12.5 | 2.5 | 0.99 | 2.99 | 12.5 | 17 | 50 | 12.5 | 0.009 | 0.1 | 1 | 0.25 | 0.9 | to 5 ml |
| Piroxicam | 20 | 2.5 | 0.99 | 2.99 | 12.5 | 17 | 50 | 12.5 | 0.009 | 0.1 | 1 | 0.25 | 0.9 | to 5 ml |
| Tenoxicam | 20 | 2.5 | 0.99 | 2.99 | 12.5 | 17 | 50 | 12.5 | 0.009 | 0.1 | 1 | 0.25 | 0.9 | to 5 ml |
| Aspirin | 500 | 2.5 | 0.75 | 2.75 | 12.5 | 17 | 50 | 12.5 | 0.009 | 0.1 | 1 | 0.25 | 0.9 | to 5 ml |
| Paracetamol | 500 | 2.5 | 0.75 | 2.75 | 12.5 | 17 | 50 | 12.5 | 0.009 | 0.1 | 1 | 0.25 | 0.9 | to 5 ml |

Triptans
Tablet Formulae (mg/tab)

| | | | | Pregelatinised | | | Magnesium | | Tablet wt |
|---|---|---|---|---|---|---|---|---|---|
| | | Triprolidine HCl | Lactose | Maize Starch | Maize Starch | Ac-di-sol | Stearate | Plasdone K-29-32 | (mg/) |
| Sumatriptan | 50 | 2.5 | 145.5 | 12 | 48 | 25 | 2 | 15 | 300 |
| Zolmitriptan | 2.5 | 2.5 | 193 | 12 | 48 | 25 | 2 | 15 | 300 |

Lozenge Formulae (mg/loz)

| | | | | | | Liquid | Liquid | | | | |
|---|---|---|---|---|---|---|---|---|---|---|---|
| | | | | Magnesium | Calcium | Glucose (sol | Sugar (sol | | Water | Plasdone | Lozenge |
| | | Triprolidine HCl | Aerosil | stearate | Carbonate | contents) | contents) | Flavour | (ml) | K29-32 | wt (mg/) |
| Sumatriptan | 50 | 2.5 | 0.05 | 0.249 | 50 | 850 | 1342 | 7.05 | 47 | 1..5 | 2350 |
| Zolmitriptan | 2.5 | 2.5 | 0.05 | 0.249 | 10 | 1020 | 1196 | 7.05 | 47 | 1.5 | 2350 |

Syrup Formulae (mg/5 ml)

| | | | | Liquid | Hy- | | | Sodium | Fla- | Ethanol | Levo- | Domiphen | | |
|---|---|---|---|---|---|---|---|---|---|---|---|---|---|---|
| | | Triprolidine | Glycerol | Sucrose | droxyethyl | Citric | Sodium | Sac- | vour | 96% | men- | Hydro- | Col- | |
| | | HCl | (ml) | (ml) | cellulose | Acid | Citrate | charin | (ml) | (ml) | thol | bromide | our | Water |
| Sumitriptan | 50 | 2.5 | 0.95 | 2.8 | 12.5 | 17 | 50 | 12.5 | 0.009 | 0.1 | 1 | 0.25 | | |
| Zoimitriptan | 2.5 | 2.5 | 0.8 | 3.0 | 12.5 | 17 | 50 | 12.5 | 0.009 | 0.1 | 1 | 0.25 | | |

Antivirals
Tablet Formulae (mg/tab)

| | | | | Pregelatinised | | | Magnesium | | Tablet wt |
|---|---|---|---|---|---|---|---|---|---|
| | | Triprolidine HCl | Lactose | Maize Starch | Maize Starch | Ac-di-sol | Stearate | Plasdone K-29-32 | (mg/) |
| Amantadine | 100 | 2.5 | 195.5 | 12 | 48 | 25 | 2 | 15 | 400 |
| Aciclovir | 200 | 2.5 | 95.5 | 12 | 48 | 25 | 2 | 15 | 400 |
| Famciclovir | 250 | 2.5 | 95.5 | 12 | 48 | 25 | 2 | 15 | 450 |

Lozenge Formulae (mg/loz)

| | | | | | | Liquid | Liquid | | | | |
|---|---|---|---|---|---|---|---|---|---|---|---|
| | | | | Magnesium | Calcium | Glucose (sol | Sugar (sol | | Water | Plasdone | Lozenge |
| | | Triprolidine HCl | Aerosil | stearate | Carbonate | contents) | contents) | Flavour | (ml) | K29-32 | wt (mg/) |
| Amantadine | 100 | 2.5 | 0.05 | 0.249 | 150 | 800 | 1341 | 7.05 | 47 | 1.5 | 2350 |
| Aciclovir | 200 | 2.5 | 0.05 | 0.249 | 150 | 700 | 1241 | 7.05 | 47 | 1.5 | 2350 |
| Famciclovir | 250 | 2.5 | 0.05 | 0.249 | 150 | 650 | 1191 | 7.05 | 47 | 1.5 | 2350 |

Syrup Formulae (mg/5 ml)

| | | | | Liquid | Hy- | | | Sodium | Fla- | Ethanol | Levo- | Domiphen | | |
|---|---|---|---|---|---|---|---|---|---|---|---|---|---|---|
| | | Triprolidine | Glycerol | Sucrose | droxyethyl | Citric | Sodium | Sac- | vour | 96% | men- | Hydro- | Col- | |
| | | HCl | (ml) | (ml) | cellulose | Acid | Citrate | charin | (ml) | (ml) | thol | bromide | our | Water |
| Amantadine | 100 | 2.5 | 0.9 | 3.0 | 12.5 | 17 | 50 | 12.5 | 0.009 | 0.1 | 1 | 0.25 | | |
| Aciclovir | 200 | 2.5 | 0.9 | 2.9 | 12.5 | 17 | 50 | 12.5 | 0.009 | 0.1 | 1 | 0.25 | | |
| Famciclovir | 250 | 2.5 | 0.9 | 2.8 | 12.5 | 17 | 50 | 12.5 | 0.009 | 0.1 | 1 | 0.25 | | |

Allergy
Tablet Formulae (mg/tab)

| | | | | Pregelatinised | | | Magnesium | | Tablet wt |
|---|---|---|---|---|---|---|---|---|---|
| | | Triprolidine HCl | Lactose | Maize Starch | Maize Starch | Ac-di-sol | Stearate | Plasdone K-29-32 | (mg/) |
| Acrivastine | 8 | 2.5 | 187.5 | 12 | 48 | 25 | 2 | 15 | 300 |
| Cetirizine | 10 | 2.5 | 185.5 | 12 | 48 | 25 | 2 | 15 | 300 |
| Loratadine | 10 | 2.5 | 185.5 | 12 | 48 | 25 | 2 | 15 | 300 |
| Fexofenadine | 120 | 2.5 | 75.5 | 12 | 48 | 25 | 2 | 15 | 300 |
| Terfenadine | 60 | 2.5 | 145.5 | 12 | 48 | 25 | 2 | 15 | 300 |
| Betamethasone | 5 | 2.5 | 190.5 | 12 | 48 | 25 | 2 | 15 | 300 |
| Clemastine | 1 | 2.5 | 194.5 | 12 | 48 | 25 | 2 | 15 | 300 |
| Bropheniramine | 8 | 2.5 | 187.5 | 12 | 48 | 25 | 2 | 15 | 300 |
| Chlorpheniramine | 4 | 2.5 | 191.5 | 12 | 48 | 25 | 2 | 15 | 300 |

Lozenge Formulae (mg/loz)

| | | | | | | Liquid | Liquid | | | | |
|---|---|---|---|---|---|---|---|---|---|---|---|
| | | Triprolidine | | Magnesium | Calcium | Glucose (sol | Sugar (sol | | Water | Plasdone | Lozenge |
| | | HCl | Aerosil | stearate | Carbonate | contents) | contents) | Flavour | (ml) | K29-32 | wt (mg/) |
| Acrivastine | 8 | 2.5 | 0.05 | 0.249 | 10 | 1010 | 1264 | 7.05 | 47 | 1..5 | 2350 |
| Cetirizine | 10 | 2.5 | 0.05 | 0.249 | 10 | 1010 | 1262 | 7.05 | 47 | 1.5 | 2350 |
| Loratadine | 10 | 2.5 | 0.05 | 0.249 | 10 | 1010 | 1262 | 7.05 | 47 | 1.5 | 2350 |
| Fexofenadine | 120 | 2.5 | 0.05 | 0.249 | 100 | 900 | 1172 | 7.05 | 47 | 1.5 | 2350 |
| Terfenadine | 60 | 2.5 | 0.05 | 0.249 | 50 | 1000 | 1182 | 7.05 | 47 | 1.5 | 2350 |
| Betamethasone | 5 | 2.5 | 0.05 | 0.249 | 10 | 1010 | 1267 | 7.05 | 47 | 1.5 | 2350 |
| Clemastine | 1 | 2.5 | 0.05 | 0.249 | 10 | 1010 | 1273 | 7.05 | 47 | 1.5 | 2350 |
| Bropheniramine | 8 | 2.5 | 0.05 | 0.249 | 10 | 1010 | 1264 | 7.05 | 47 | 1.5 | 2350 |
| Chlorpheniramine | 4 | 2.5 | 0.05 | 0.249 | 10 | 1010 | 1260 | 7.05 | 47 | 1.5 | 2350 |

Syrup Formulae (mg/5 ml)

| | | | Glyc- | Liquid | Hy- | | | Sodium | Fla- | Ethanol | Levo- | Domiphen | | |
|---|---|---|---|---|---|---|---|---|---|---|---|---|---|---|
| | | Triprolidine | erol | Sucrose | droxyethyl | Citric | Sodium | Sac- | vour | 96% | men- | Hydro- | Col- | |
| | | HCl | (ml) | (ml) | cellulose | Acid | Citrate | charin | (ml) | (ml) | thol | bromide | our | Water |
| Acrivastine | 8 | 2.5 | 0.9 | 2.9 | 12.5 | 17 | 50 | 12.5 | 0.009 | 0.1 | 1 | 0.25 | 0.9 | to 5 ml |
| Cetirizine | 10 | 2.5 | 0.8 | 2.8 | 12.5 | 17 | 50 | 12.5 | 0.009 | 0.1 | 1 | 0.25 | 0.9 | to 5 ml |
| Loratadine | 10 | 2.5 | 0.99 | 2.99 | 12.5 | 17 | 50 | 12.5 | 0.009 | 0.1 | 1 | 0.25 | 0.9 | to 5 ml |
| Fexofenadine | 120 | 2.5 | 0.99 | 2.99 | 12.5 | 17 | 50 | 12.5 | 0.009 | 0.1 | 1 | 0.25 | 0.9 | to 5 ml |
| Terfenadine | 60 | 2.5 | 0.95 | 2.95 | 12.5 | 17 | 50 | 12.5 | 0.009 | 0.1 | 1 | 0.25 | 0.9 | to 5 ml |
| Beclamethasone | 5 | 2.5 | 0.9 | 2.8 | 12.5 | 17 | 50 | 12.5 | 0.009 | 0.1 | 1 | 0.25 | 0.9 | to 5 ml |
| Clemastine | 1 | 2.5 | 0.95 | 2.95 | 12.5 | 17 | 50 | 12.5 | 0.009 | 0.1 | 1 | 0.25 | 0.9 | to 5 ml |
| Bropheniramine | 8 | 2.5 | 0.95 | 2.95 | 12.5 | 17 | 50 | 12.5 | 0.009 | 0.1 | 1 | 0.25 | 0.9 | to 5 ml |
| Chlorphiniramine | 4 | 2.5 | 0.95 | 2.95 | 12.5 | 17 | 50 | 12.5 | 0.009 | 0.1 | 1 | 0.25 | 0.9 | to 5 ml |

Cough/Cold
Tablet Formulae (mg/tab)

| | | | | Pregelatinised | | | Magnesium | | Tablet wt |
|---|---|---|---|---|---|---|---|---|---|
| | | Triprolidine HCl | Lactose | Maize Starch | Maize Starch | Ac-di-sol | Stearate | Plasdone K-29-32 | (mg/) |
| Ambroxol | 30 | 2.5 | 165.5 | 12 | 48 | 25 | 2 | 15 | 300 |
| Guaiphenesin | 100 | 2.5 | 195.5 | 12 | 48 | 25 | 2 | 15 | 400 |
| Dextromethorphan | 10 | 2.5 | 185.5 | 12 | 48 | 25 | 2 | 15 | 300 |
| Menthol | 10 | 2.5 | 185.5 | 12 | 48 | 25 | 2 | 15 | 300 |
| Phenylpropanolamine | 12.5 | 2.5 | 183 | 12 | 48 | 25 | 2 | 15 | 300 |

Lozenge Formulae (mg/loz)

| | | | | | | Liquid | Liquid | | | | |
|---|---|---|---|---|---|---|---|---|---|---|---|
| | | Triprolidine | | Magnesium | Calcium | Glucose (sol | Sugar (sol | | Water | Plasdone | Lozenge |
| | | HCl | Aerosil | stearate | Carbonate | contents) | contents) | Flavour | (ml) | K29-32 | wt (mg/) |
| Ambroxol | 30 | 2.5 | 0.05 | 0.249 | 10 | 1000 | 1262 | 7.05 | 47 | 1.5 | 2350 |
| Guaiphenesin | 100 | 2.5 | 0.05 | 0.249 | 75 | 825 | 1292 | 7.05 | 47 | 1.5 | 2350 |
| Dextromethorphan | 10 | 2.5 | 0.05 | 0.249 | 10 | 980 | 1262 | 7.05 | 47 | 1.5 | 2350 |
| Menthol | 10 | 2.5 | 0.05 | 0.249 | 10 | 980 | 1262 | 7.05 | 47 | 1.5 | 2350 |
| Phenylpropanolamine | 12.5 | 2.5 | 0.05 | 0.249 | 10 | 978 | 1262 | 7.05 | 47 | 1.5 | 2350 |

Syrup Formulae (mg/5 ml)

| | | | Glyc- | Liquid | Hy- | | | Sodium | Fla- | Ethanol | Levo- | Domiphen | | |
|---|---|---|---|---|---|---|---|---|---|---|---|---|---|---|
| | | Triprolidine | erol | Sucrose | droxyethyl | Citric | Sodium | Sac- | vour | 96% | men- | Hydro- | Col- | |
| | | HCl | (ml) | (ml) | cellulose | Acid | Citrate | charin | (ml) | (ml) | thol | bromide | our | Water |
| Ambroxol | 30 | 2.5 | 0.9 | 2.9 | 12.5 | 17 | 50 | 12.5 | 0.009 | 0.1 | 1 | 0.25 | 0.9 | to 5 ml |
| Guaiphenesin | 100 | 2.5 | 0.8 | 2.8 | 12.5 | 17 | 50 | 12.5 | 0.009 | 0.1 | 1 | 0.25 | 0.9 | to 5 ml |
| Dextro- | 10 | 2.5 | 0.99 | 2.99 | 12.5 | 17 | 50 | 12.5 | 0.009 | 0.1 | 1 | 0.25 | 0.9 | to 5 ml |
| methorphan | | | | | | | | | | | | | | |
| Menthol | 10 | 2.5 | 0.99 | 2.99 | 12.5 | 17 | 50 | 12.5 | 0.009 | 0.1 | 1 | 0.25 | 0.9 | to 5 ml |
| Phenyl- | 12.5 | 2.5 | 0.95 | 2.95 | 12.5 | 17 | 50 | 12.5 | 0.009 | 0.1 | 1 | 0.25 | 0.9 | to 5 ml |
| propanolamine | | | | | | | | | | | | | | |

Upper Respiratory
Tablet Formulae (mg/tab)

| | | | | Pregelatinised | | | Magnesium | | Tablet wt |
|---|---|---|---|---|---|---|---|---|---|
| | | Triprolidine HCl | Lactose | Maize Starch | Maize Starch | Ac-di-sol | Stearate | Plasdone K-29-32 | (mg/) |
| Benzoczine | 10 | 2.5 | 185.5 | 12 | 48 | 25 | 2 | 15 | 300 |
| Lignocaine | 10 | 2.5 | 185.5 | 12 | 48 | 25 | 2 | 15 | 300 |
| Hexylresourcinol | 2.5 | 2.5 | 193 | 12 | 48 | 25 | 2 | 15 | 300 |
| Tyrothricin | 1 | 2.5 | 194.5 | 12 | 48 | 25 | 2 | 15 | 300 |
| Dichlobenzyl alcohol | 1.2 | 2.5 | 194.7 | 12 | 48 | 25 | 2 | 15 | 300 |
| Amyl methyl cresol | 0.6 | 2.5 | 194.1 | 12 | 48 | 25 | 2 | 15 | 300 |
| Cetyl pyridinium | 2 | 2.5 | 193.5 | 12 | 48 | 25 | 2 | 15 | 300 |
| chloride | | | | | | | | | |

Lozenge Formulae (mg/loz)

| | | | | Mag- | | Liquid | Liquid | | | | |
|---|---|---|---|---|---|---|---|---|---|---|---|
| | Triprolidine | Triprolidine | | nesium | Calcium | Glucose (sol | Sugar (sol | | Water | Plasdone | Lozenge |
| | HCl | HCl | Aerosil | stearate | Carbonate | contents) | contents) | Flavour | (ml) | K29-32 | wt (mg/) |
| Benzoczine | 10 | 2.5 | 0.05 | 0.249 | 10 | 980 | 1262 | 7.05 | 47 | 1.5 | 2350 |
| Lignocaine | 10 | 2.5 | 0.05 | 0.249 | 10 | 980 | 1262 | 7.05 | 47 | 1.5 | 2350 |
| Hexylresourcinol | 2.5 | 2.5 | 0.05 | 0.249 | 10 | 987.5 | 1262 | 7.05 | 47 | 1.5 | 2350 |
| Tyrothricin | 1 | 2.5 | 0.05 | 0.249 | 10 | 989 | 1262 | 7.05 | 47 | 1.5 | 2350 |
| Dichlobenzyl | 1.2 | 2.5 | 0.05 | 0.249 | 10 | 988.8 | 1262 | 7.05 | 47 | 1.5 | 2350 |
| alcohol | | | | | | | | | | | |
| Amyl methyl cresol | 0.6 | 2.5 | 0.05 | 0.249 | 10 | 989.4 | 1262 | 7.05 | 47 | 1.5 | 2350 |
| Cetyl pyridinium | 2 | 2.5 | 0.05 | 0.249 | 10 | 988 | 1262 | 7.05 | 47 | 1.5 | 2350 |
| chloride | | | | | | | | | | | |

Syrup Formulae (mg/5 ml)

| | | | Glyc- | Liquid | Hy- | | | Sodium | Fla- | Ethanol | Levo- | Domiphen | | |
|---|---|---|---|---|---|---|---|---|---|---|---|---|---|---|
| | | Triprolidine | erol | Sucrose | droxyethyl | Citric | Sodium | Sac- | vour | 96% | men- | Hydro- | Col- | |
| | | HCl | (ml) | (ml) | cellulose | Acid | Citrate | charin | (ml) | (ml) | thol | bromide | our | Water |
| Benzoczine | 10 | 2.5 | 0.9 | 2.9 | 12.5 | 17 | 50 | 12.5 | 0.009 | 0.1 | 1 | 0.25 | 0.9 | to 5 ml |
| Lignocaine | 10 | 2.5 | 0.8 | 2.8 | 12.5 | 17 | 50 | 12.5 | 0.009 | 0.1 | 1 | 0.25 | 0.9 | to 5 ml |
| Hexylresourcinol | 2.5 | 2.5 | 0.99 | 2.99 | 12.5 | 17 | 50 | 12.5 | 0.009 | 0.1 | 1 | 0.25 | 0.9 | to 5 ml |
| Tyrothricin | 1 | 2.5 | 0.99 | 2.99 | 12.5 | 17 | 50 | 12.5 | 0.009 | 0.1 | 1 | 0.25 | 0.9 | to 5 ml |
| Dichlobenzyl | 1.2 | 2.5 | 0.95 | 2.95 | 12.5 | 17 | 50 | 12.5 | 0.009 | 0.1 | 1 | 0.25 | 0.9 | to 5 ml |
| alcohol | | | | | | | | | | | | | | |
| Amyl methyl | 0.6 | 2.5 | 1.0 | 3.0 | 12.5 | 17 | 50 | 12.5 | 0.009 | 0.1 | 1 | 0.25 | 0.9 | to 5 ml |
| cresol | | | | | | | | | | | | | | |
| Cetyl pyridinium | 2 | 2.5 | 0.99 | 2.9 | 12.5 | 17 | 50 | 12.5 | 0.009 | 0.1 | 1 | 0.25 | 0.9 | to 5 ml |
| chloride | | | | | | | | | | | | | | |

Anxiety
Tablet Formulae (mg/tab)

| | | | | Pregelatinised | | | Magnesium | | Tablet wt |
|---|---|---|---|---|---|---|---|---|---|
| | | Triprolidine HCl | Lactose | Maize Starch | Maize Starch | Ac-di-sol | Stearate | Plasdone K-29-32 | (mg/) |
| Propanolol | 10 | 2.5 | 185.5 | 12 | 48 | 25 | 2 | 15 | 300 |
| Propanolo | 20 | 2.5 | 175.5 | 12 | 48 | 25 | 2 | 15 | 300 |
| Propanolol | 40 | 2.5 | 155.5 | 12 | 48 | 25 | 2 | 15 | 300 |

Lozenge Formulae (mg/loz)

| | | | | | | Liquid | Liquid | | | | |
|---|---|---|---|---|---|---|---|---|---|---|---|
| | | | | Magnesium | Calcium | Glucose (sol | Sugar (sol | | Water | Plasdone | Lozenge |
| | | Triprolidine HCl | Aerosil | stearate | Carbonate | contents) | contents) | Flavour | (ml) | K29-32 | wt (mg/) |
| Propanolol | 10 | 2.5 | 0.05 | 0.249 | 10 | 980 | 1262 | 7.05 | 47 | 1..5 | 2350 |
| Propanolo | 20 | 2.5 | 0.05 | 0.249 | 10 | 970 | 1262 | 7.05 | 47 | 1.5 | 2350 |
| Propanolol | 40 | 2.5 | 0.05 | 0.249 | 10 | 950 | 1262 | 7.05 | 47 | 1.5 | 2350 |

Syrup Formulae (mg/5 ml)

| | | | | Liquid | | | | Sodium | Fla- | Ethanol | Levo- | Domiphen | | |
|---|---|---|---|---|---|---|---|---|---|---|---|---|---|---|
| | | Triprolidine | Glycerol | Sucrose | Hydroxyethyl | Citric | Sodium | Sac- | vour | 96% | men- | Hydro- | Col- | |
| | | HCl | (ml) | (ml) | cellulose | Acid | Citrate | charin | (ml) | (ml) | thol | bromide | our | Water |
| Propanolol | 10 | 2.5 | 0.9 | 3.0 | 12.5 | 17 | 50 | 12.5 | 0.009 | 0.1 | 1 | 0.25 | | |
| Propanolol | 20 | 2.5 | 0.9 | 2.9 | 12.5 | 17 | 50 | 12.5 | 0.009 | 0.1 | 1 | 0.25 | | |
| Propanolol | 40 | 2.5 | 0.9 | 2.8 | 12.5 | 17 | 50 | 12.5 | 0.009 | 0.1 | 1 | 0.25 | | |

TABLE 2
Loughborough Sleep Log: Awoke Very Refreshed
or Refreshed Responses

| | Day of Testing | | | | | |
|---|---|---|---|---|---|---|
| | Monday | | Tuesday | | Wednesday | |
| Dose | N | % | n | % | n | % |
| Placebo | 10 | 15.2 | 10 | 16.4 | 11 | 18.3 |
| 2.5 mg TRP•HCl•H₂O | 14 | 23 | 14 | 23 | 16 | 25.8 |
| 5 mg TRP•HCl•H₂O | 7 | 11.5 | 5 | 8.2 | 9 | 14.8 |

Similarly, table 3 shows corresponding additional data in connection with data set (b).

TABLE 3
Loughborough Sleep Log: Last Night I Slept Extremely
Well or Very Well Responses

| | Day of Testing | | | | | |
|---|---|---|---|---|---|---|
| | Monday | | Tuesday | | Wednesday | |
| Dose | N | % | n | % | n | % |
| Placebo | 11 | 18 | 12 | 22.2 | 13 | 24.1 |
| 2.5 mg TRP•HCl•H₂O | 24 | 41.4 | 23 | 41.8 | 22 | 37.9 |
| 5 mg TRP•HCl•H₂O | 30 | 50.9 | 17 | 28.8 | 24 | 39.3 |

TABLE 4
Karolinska 9-point scale
(a) I feel extremely alert, very alert or alert

| | Day of Testing | | | | | |
|---|---|---|---|---|---|---|
| | Monday | | Tuesday | | Wednesday | |
| Dose | n | % | n | % | n | % |
| Placebo | 9 | 13.6 | 14 | 23.0 | 11 | 17.2 |
| 2.5 mg TRP•HCl•H₂O | 13 | 21.3 | 13 | 21.3 | 13 | 21.0 |
| 5 mg TRP•HCl•H₂O | 4 | 6.3 | 6 | 9.5 | 11 | 17.5 |

TABLE 5
(b) I feel (i) sleepy, [and need to make] some effort or
(ii) very sleepy, a great effort to keep awake

| | Day of Testing | | | | | |
|---|---|---|---|---|---|---|
| | Monday | | Tuesday | | Wednesday | |
| Dose | n | % | n | % | n | % |
| Placebo | 8 | 12.1 | 10 | 16.4 | 9 | 14.1 |
| 2.5 mg TRP•HCl•H₂O | 7 | 11.5 | 8 | 13.1 | 4 | 6.5 |
| 5 mg TRP•HCl•H₂O | 8 | 12.5 | 11 | 17.5 | 8 | 12.7 |

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. The use of triprolidine or a salt or hydrate thereof, in combination with at least one further active pharmaceutical agent, as active ingredient of an aid to waking refreshed after sleeping.

2. The use of triprolidine or a salt or hydrate thereof, in combination with at least one further active pharmaceutical agent, as active ingredient for the preparation of a composition for enabling an individual to wake refreshed after sleeping.

3. The use of triprolidine or a salt or hydrate thereof, in combination with at least one further active pharmaceutical agent, as active ingredient for the preparation of a medicament for enabling an individual to wake refreshed after sleeping.

4. The use of triprolidine or a salt or hydrate thereof, in combination with at least one further active pharmaceutical agent, for the preparation of a sleep aid which also enables an individual to wake refreshed after sleeping.

5. The use of triprolidine or a salt or hydrate thereof, in combination with at least one further active pharmaceutical agent, as active ingredient of a sleep aid which also enables an individual to wake refreshed after sleeping.

6. The use of triprolidine or a salt or hydrate thereof, in combination with at least one further active pharmaceutical agent, as active ingredient for the preparation of a medicament for the treatment or prevention of a sleep disorder which also enables an individual to wake refreshed after sleeping.

7. Use of triprolidine or a salt or hydrate thereof, in combination with at least one further active pharmaceutical agent, as active ingredient in the manufacture of a composition for the treatment of sleep disorders.

8. The use of triprolidine or a salt or hydrate thereof, in combination with at least one further active pharmaceutical agent, as active ingredient in the manufacture of a composition for inducing, prolonging and/or enhancing sleep and/or sleep quality.

9. A method for the treatment or prevention of grogginess, drowsiness or lethargy on waking from sleep in a mammal comprising the administration to the mammal in need thereof of a non-toxic effective dose of triprolidine or a salt or hydrate thereof in combination with at least one further active pharmaceutical agent prior to the desired sleeping time.

10. A method for enabling an individual to wake refreshed after sleeping comprising the administration to the individual in need thereof and prior to the desired sleeping time of a non-toxic effective dose of triprolidine or a salt or hydrate thereof in combination with at least one further active pharmaceutical agent.

11. A method for aiding an individual's sleep and for also enabling the individual to subsequently wake refreshed after sleeping comprising the administration to the individual in need thereof and prior to the desired sleeping time of a non-toxic effective dose of triprolidine or a salt or hydrate thereof in combination with at least one further active pharmaceutical agent.

12. A method of treating sleep of a person suffering from a sleep disorder, which method comprises administration of an effective dose of triprolidine or a salt or hydrate thereof, in combination with at least one further active pharmaceutical agent, as active ingredient to such a person.

13. A method for inducing, prolonging and/or enhancing sleep, which method comprises administration of an effective dose of triprolidine or a salt or hydrate thereof, in combination with at least one further active pharmaceutical agent, as active ingredient to a person desirous of achieving sleep.

14. A waking refreshed aid comprising triprolidine or a salt or hydrate thereof, in combination with at least one further active pharmaceutical agent, as active ingredient in association with a pharmaceutically acceptable carrier therefor and instructions for administration thereof at or just before the desired sleeping time.

15. A pharmaceutical formulation for the treatment or prevention of grogginess, drowsiness or lethargy on waking after sleeping, comprising triprolidine or a salt or hydrate thereof, in combination with at least one further active pharmaceutical agent, as active ingredient in association with a pharmaceutically acceptable carrier therefor and instructions for administration thereof at or just before the desired sleeping time.

16. A pharmaceutical formulation for enabling an individual to wake more refreshed after sleeping, comprising triprolidine or a salt or hydrate thereof, in combination with at least one further active pharmaceutical agent, as active ingredient in association with a pharmaceutically acceptable carrier therefor and instructions for administration thereof at or just before the desired sleeping time.

17. The use of triprolidine or a salt or hydrate thereof as claimed in claim 1, wherein the said at least one further active pharmaceutical agent is intended to be used in the treatment of a condition having sleep disorder as a symptom or potential symptom.

18. The use of triprolidine or a salt or hydrate thereof as claimed in claim 1, wherein the said at least one further active pharmaceutical agent is selected from: an active agent used in the treatment of pain relief, migraines, allergies, colds, flu, coughs or anxiety; an active agent used as an anaesthetic, antiviral agent, antidepressive agent, decongestant or disinfectant; or an active agent used in women's health.

19. The use of triprolidine or a salt or hydrate thereof as claimed in claim 1, wherein the said at least one further active agent is independently selected from any one or more of the following agents or their active salts or hydrates: Ibuprofen, Fluribiprofen, Ketoprofen, Aspirin, Paracetamol, Aceclofenac, Codeine, Naproxen, Indomethacin, Diclofenac, Cox II, Meloxicam, Nitric oxide, Caffeine, Acrivastine, Cetirizine, Loratadine, Fexofenadine, Terfenadine, Beclomethasone, Hydrocortisone, Triptan, Almotriptan, Rizatriptan, Naratriptan, Sumatriptan, Zolmatriptan, Domperidone, Acetylcysteine, Menthol, Ambroxol, Carbocisteine, Dextromethorphan, Guaiphenesin, Ipecacuanha, Phenylpropanolamine, Liquorice, Marshmallow, Squill, Honey, Glycerine, Aniseed, Benzocaine, Lidocaine, Amantadine, Aciclovir, Famciclovir, Ganciclovir, Rimantadine, Penciclovir, Tribavirin, Valaciclovir, Neuraminidase inhibitors, Zanamir, Oseltamir, Benzalkonium chloride, Cetylpyridinium chloride, Dichlorobenzyl alcohol, Amylmetacresol, Dequalinium chloride, Hexylresorcinol, Eucalyptus oil, Thymol, Calamine, Propranalol, Chamomile, Hops, Passion flower, Valarian, Melatonin, Eucalyptus, Phenylepherine, Pseudoephedrine, Cranberry and Bisphosphonates.

20. The use of triprolidine as a salt or hydration thereof as claimed in claim 19, wherein the said active pharmaceutical agent is independently selected from any one or more of the following agents or their active salts or hydrates Ibuprofen, Fluribiprofen, Cox II such as meloxicam, triptans, Domperidone, Ambroxol, Dextromethorphan, Guaiphenesin, Lidocaine, Amiantadine, Hexylresorcinol, dcba, amc, Propranalol, pseudoephedrine and Bisphosphonates or a pharmaceutically acceptable salt of any of the foregoing.

21. The use of triprolidine or a salt or hydrate thereof as claimed in claim 1, wherein the further active pharmaceutical agent may be combined with triprolidine in a single dosage form or in a pharmaceutical pack containing at least two dose forms, one being triprolidine and the other being the said further active pharmaceutical agent.

22. The use of triprolidine or a salt or hydrate thereof as claimed in claim 1, wherein the said pack includes instructions on how to take the combination of triprolidine with the said further agent.

23. The use of triprolidine or a salt or hydrate thereof as claimed in claim 1, wherein the dosage of the said further pharmaceutically active agent is one suitable for the treatment selected.

24. The use as claimed in claim 1, wherein the dose of triprolidine administered to the user prior to sleeptime is between 0.01 mg and 20 mg.

25. The use as claimed in claim 1, wherein the dose of triprolidine administered to the user before sleeptime is up to 20 mg.

26. The use as claimed in claim 1, wherein the said further active pharmaceutical agent may include, without limitation, antacids, analgesics, anti-inflammatories, antibiotics, laxatives, anorexics, antiasthmatics, antidiuretics, antiflatulents, antimigraine agents, antispasmodics, additional sedatives, antihyperactives, tranquilizers, antihistamines, decongestants, betablockers, antidepressives, hormones and combinations thereof.

27. The use as claimed in claim 1, wherein the said dosage forms may be combined into a combined dosage form for simultaneous administration.

28. The method as claimed in claim 9, wherein the dose of active ingredient of triprolidine administered is between 0.01 and 20 mg.

29. The method as claimed in claim 9 wherein the dose of active ingredient of triprolidine administered is up to 20 mg.

30. The pharmaceutical formulation as claimed in claim 15, wherein the instructions for administration instruct a single dose comprising active ingredient of triprolidine of up to 20 mg prior to sleeptime.

31. The pharmaceutical formulation as claimed in claim 15, wherein the instructions for administration instruct a single dose comprising active ingredient of triprolidine of between 0.01 and 20 mg prior to sleeptime.

32. A waking refreshed aid as claimed in claim 14, wherein the instructions for administration instruct a single dose of the triprolidine active ingredient of up to 20 mg prior to sleeptime.

33. A waking refreshed aid as claimed in claim 14, wherein the instructions for administration instruct a single dose comprising the active ingredient triprolidine of between 0.01 and 20 mg prior to sleeptime.

34. A method as claimed in claim 9, wherein the triprolidine is in the form of triprolidine hydrochloride.

35. A method as claimed in claim 9, wherein the person is suffering from a sleep disorder.

36. A method as claimed in claim 9, wherein the person is not suffering from a sleep disorder but is desirous of achieving a feeling of waking refreshed upon waking.

37. A method as claimed in claim 9, wherein the active ingredients are administered orally, nasally, optically, rectally, pulmonarily, transdermally or sub-lingually.

38. A method as claimed in claim 9, wherein the active ingredients are administered in the form of a tablet, capsule, drink, lozenge, drops, emulsion, dry powder, suspension, pastille, patch, suppository, syrup, consumable film such as a buccal wafer, sub-lingual spray or nasal spray.

39. A method as claimed in claim 9, wherein the active ingredients are administered to the mucous membranes of the nasal cavity.

40. A method as claimed in claim 9, wherein the active ingredients are administered as a solution or suspension spray or as a powder.

41. A method as claimed in claim 9 in which the active ingredients are administered between 1 minute and 2 hours prior to sleeptime.

42. Use as claimed in claim 1, wherein the triprolidine is in the form of triprolidine hydrochloride.

43. Use as claimed in claim 1, wherein the composition is for oral administration.

44. Use as claimed in claim 1, wherein the composition is in the form of a tablet, capsule, drink, lozenge, drops, emulsion, dry powder, suspension, pastille, patch, suppository, syrup, consumable film such as a buccal wafer, sub-lingual spray or nasal spray.

45. Use as claimed in claim 1, wherein the composition is for administration to the mucous membranes of the nasal cavity.

46. Use as claimed in claim 1, wherein the composition is a solution or suspension or a powder.

47. The use as claimed in claim 1, wherein the triprolidine forms the active ingredient of a formulation which contains a blend of two or more diluents, one of which may also serve as a disintegrant.

48. The use as claimed in claim 1, wherein the triprolidine forms the active ingredient of a formulation, which comprises a saccharide diluent.

49. The use as claimed in claim 48, wherein the triprolidine formulation further comprises a disintegrant.

50. The use as claimed in claim 49, wherein the triprolidine formulation further comprises the saccharide diluent and the disintegrant in the ratio of 1-10 parts by weight sacaharide diluent to 1 part by weight of disintegrant.

51. The use as claimed in claim 49, wherein the saccharide diluent is lactose, and the disintegrant is croscarmellose sodium.

52. The use as claimed in claim 47, wherein the triprolidine formulation further comprises a lubricant.

53. The use as claimed in claim 52, wherein the lubricant is magnesium stearate.

54. The use as claimed in claim 47, wherein the triprolidine formulation is formed with a coating of a hydrophilic polymer.

55. The use as claimed in claim 54, wherein the hydrophilic polymer is a methylated cellulose derivative.

56. The use as claimed in claim 47, which is free of ingredients intended or effective to sustain or prolong release of the active ingredients.

57. The use as claimed in claim 1, wherein the dosage of the said further pharmaceutically active agent is one suitable for the treatment selected.

58. The use as claimed in claim 1, wherein a single dosage form of said pharmaceutically active agent is in the range 0.1 mg-2000 mg.

59. A method of manufacturing a formulation as claimed in claim 45, which involves direct compression of the ingredients into a tablet without an intermediate granulation stage.

60-71. (canceled)
